(19) United States
(12) Patent Application Publication
(10) Pub. No.: US 2010/0030152 A1
(43) Pub. Date: Feb. 4, 2010

(54) Multi Injection Microneedle Therapy System

(75) Inventors: Man Kyu Lee, Gyeonggi-do (KR); Byeong Jun Kim, Kangwon-Do (KR)

Correspondence Address: INTELLECTUAL PROPERTY LAW GROUP LLP, 12 SOUTH FIRST STREET, SUITE 1205, SAN JOSE, CA, 95113, US

(73) Assignee: MCURE CO., LTD., Wonju-si, Kangwon-Do (KR)

(21) Appl. No.: 12/516,349

(22) PCT Filed: May 29, 2008
(86) PCT No.: PCT/KR08/03011

§ 371 Date: May 26, 2009

(30) Foreign Application Priority Data
May 31, 2007 (KR) 10-2007-0053282

(51) Int. Cl. A61M 5/32

(52) U.S. Cl. 604/131

(57) ABSTRACT

The present invention relates to a microneedle therapy system for skin care, hair loss treatment, obesity treatment, and the like. Provided is a multi-injection microneedle therapy system which implements a new treatment device and method capable of inserting a number of needles to create holes on the human skin and simultaneously inject drugs by using a gun, thereby significantly reducing a procedure time, minimizing pain, and increasing drug absorption efficiency to maximize treatment effectiveness. In addition, the multi injection microneedle therapy system can perform microneedle therapy or mesotherapy treatment according to types of injected drugs and effectively perform the mesotherapy treatment by performing a number of injections for a short procedure time.

### TECHNICAL FIELD

The present invention relates to a microneedle therapy system and method for skin care, hair loss treatment, obesity treatment, and the like, and more particularly, to a multi-injection microneedle therapy system which applies a new treatment of inserting a plurality of needles into the skin to create holes on the human skin and simultaneously injecting drugs into the human skin thereby reducing a procedure time and pain during a procedure and maximizing treatment effectiveness.

### BACKGROUND ART

In general, a microneedle therapy system is for skin and hair care treatment of various conditions such as wrinkles, acre scarring, and dead skin cells. In the microneedle therapy, a method of piercing holes in the skin and applying drugs or cosmetics to the skin so as to rapidly and sufficiently permeate the skin to maximize effectiveness, a method of using needles to injure the skin just enough to begin natural healing processes and stimulate collagen production, and the like are used. This is generally applied to faces and scalps.

For example, in the aforementioned methods, hundreds to ten thousands of tiny holes are created on the skin, skin tissue injured during the creation of the tiny holes begins natural healing processes to produce a new skin tissue, medical substances are applied to the site where the holes are created, and the substances quickly permeate into the skin through the tiny holes in order to maximize effectiveness.

Unlike existing treatments (such as lasers and peel) of wrinkles or pigmentation by exfoliating the skin to regenerate, the microneedle therapy is a new concept cell recovery therapy for inducing the body to produce collagen without removing or damaging the epidermis of the skin.

In the microneedle therapy that is simple and safe and does not exhibit sides effects, as a number of needles are inserted into the skin, the injured skin tissue begins natural wound healing processes so as to form and rearrange a new dermal tissue, thereby smoothing scars and the skin to remove wrinkles and improving pigmentation, for healthy and clean skin.

In addition, hundreds of the needles create hundreds to ten thousands of micro-channels across the skin for drug delivery of active principles such as active peptides to maximize treatment effectiveness.

As a method of forming holes on the human skin, there are methods using multi roller needles and laser.

Mesotherapy is a treatment that employs multiple injections of pharmaceutical medications into the human body through needles for obesity, stretch marks, wrinkles, and hair loss and shows more accurate effects as compared with a method of taking or applying medicine.

As described above, the microneedle therapy is a treatment that creates holes on the human skin to apply drugs thereto and includes methods of using rollers, laser, guns, and the like. However, in the microneedle therapy, drugs are applied after creating the holes, so that a long procedure time is needed. In addition, since it is impossible to absorb 100 percent of the applied drugs, a large amount of drugs are needed.

In addition, in the mesotherapy, drugs are injected by using a single needle, so that a long procedure time is needed in a case of obesity treatment or hair loss treatment that needs a large number of injections, and pain must be accepted with the injections. In order to solve the problems, a method of using a microneedle is introduced. However, the conventional microneedle is large and cannot be easily mounted on an automatic injector gun (mesogun) to be used, so that the microneedle is used only for manual injections. In addition, the microneedle mesotherapy treatment performed by the manual injections may cause great pain during the procedures and is not practically frequently used.

### DISCLOSURE

### Technical Problem

The present invention provides a multi-injection microneedle therapy system which implements a new treatment device and method capable of inserting a number of needles to create holes on the human skin and simultaneously inject drugs by using a gun, thereby significantly reducing a procedure time, minimizing pain, and increasing drug absorption efficiency to maximize treatment effectiveness.

The present invention also provides a multi-injection microneedle therapy system capable of performing microneedle therapy or mesotherapy treatment according to types of injected drugs and effectively performing the mesotherapy treatment by performing a number of injections for a short procedure time.

### Technical Solution

According to an aspect of the present invention, there is provided a multi-injection microneedle therapy system including: a microneedle mount which has a number of needles to deliver drugs and is connected to a syringe, a gun which has functions of inserting the microneedle mount into the skin and injecting the drugs supplied to the syringe; and a cart which has functions of providing power for operating the gun and simultaneously controlling the operations of the gun, wherein a number of holes are created on the human skin and simultaneously the drugs are injected into the human skin by using the microneedle mount and the syringe according to the operations of the gun.

In the above aspect of the present invention, an example of the microneedle mount may include: a syringe connector connected with the syringe; a gun connector mounted on the gun; a drug delivery tube for connecting the syringe connector to the gun connector; a plurality of the needles connected to a front portion of the gun connector; and a hub for supporting the needles, wherein the drugs supplied through the syringe connector are injected through the needles of the gun connector.

In addition, the needles may be mounted to be inserted through the gun connector so as to be extended inside the drug delivery tube to be directly supplied with the drugs from the drug delivery tube.

In addition, a sealing portion for preventing drug leakage may be formed at the portion of the gun connector through which the needles penetrate.

In addition, the microneedle mount may include: a syringe connector connected with the syringe; a gun connector which has a hollow through which the drugs are injected and is mounted on the gun; a drug delivery tube for connecting the syringe connector to the gun connector; and a plurality of the needles which penetrate a front portion of the gun connector to be engaged therewith and are connected with the hollow, wherein the drugs supplied through the syringe connector are injected through the needles of the gun connector.

In addition, a sealing portion for preventing drug leakage may be formed at the portion of the gun connector through which the needles penetrate.

According to another aspect of the present invention, there is provided a microneedle therapy method including: mounting a microneedle mount having a number of needles for drug delivery on a gun having a syringe; and inserting the needles of the microneedle mount into the skin to create a number of holes by operating the gun and simultaneously inject drugs supplied to the syringe.

In the above aspect of the present invention, mesotherapy treatment may be performed according to types of the drugs supplied to the syringe.

In addition, an insertion speed and an insertion depth may be controlled by changing an operation mode of the gun according to characteristics of the patient.

### DESCRIPTION OF DRAWINGS

FIG. 1 is a perspective view illustrating a microneedle therapy system according to an embodiment of the present invention.

FIG. 2 is a cross-sectional view illustrating the microneedle therapy system according to the embodiment of the present invention.

FIG. 3 is a perspective view illustrating a microneedle therapy system according to another embodiment of the present invention.

FIG. 4 is a cross-sectional view illustrating the microneedle therapy system according to the embodiment of the present invention.

FIG. 5 is a perspective view illustrating a drug stopper applied to the microneedle therapy system according to the present invention.

FIGS. 6 to 8 are schematic views illustrating operation states of the microneedle therapy system according to the embodiment of the present invention.

### BEST MODE

The present invention basically employs a microneedle injection method capable of creating holes on the skin by using needles, injecting drugs through the needles, and simultaneously injecting drugs through a number of the needles during a single procedure. In addition, microneedle treatment which requires a short procedure time according to types of the drugs injected by using a device according to the present invention and can maximize drug absorption efficiency can be implemented. In addition, mesotherapy treatment which can perform a large number of injections for a short time can be implemented.

For example, according to the present invention, a new type microneedle and a device of inserting the microneedle is applied to the microneedle therapy and the mesotherapy treatment.

For this, after mounting a number of needles to a predetermined mount, a device that can supply drugs to the needles is prepared, and

a gun that can operate the mount on which the needles are mounted, that is, a gun for simultaneously inserting a number of needles into the human skin, and a device for operating the gun, for example, a cart for operation controls of the gun, are provided to be used for the microneedle therapy and the mesotherapy treatment according to types of the injected drugs.

Accordingly, after mounting the mount into which a number of fine needles are inserted on the gun, a linear force is exerted on the mount to simultaneously insert a number of the needles to pierce the human skin and simultaneously inject the drugs through the needles into the skin, thereby significantly reducing a procedure time and maximizing drug absorption efficiency. As described above, effectiveness of the microneedle therapy or the mesotherapy treatment can be maximized.

The cart as a control means may apply a general cart used for the microneedle therapy or the mesotherapy treatment. For example, the cart has functions of power supply, supply of power sources such as air, control of an operation speed or a mode of the gun, and the like. The cart includes: a power supply device in a type of a switched-mode power supply (SMPS); a control logic (program) for controlling a liquid crystal display (LCD), a solenoid value, and the like; a main board including a control logic for changing a mode of the gun; and a compressor for generating and providing compressed air, and the like. The cart is electrically and pneumatically connected to the gun.

In addition, the cart may include external manipulation and display means such as switches, display windows for display of operation relationships or the like, lamps for informing power ON/OFF, and the like.

The cart as described above performs functions of controlling speeds and displaying the procedure in associated with operations of the gun in addition to supplying power and air to the gun.

For example, when power is supplied to the cart, an alternating current (AC) power is supplied to operate the compressor and the SMPS, and the LCD and the insertion speed of the needles are controlled by controlling the window of the LCD of the body cart, that is, pressing the mode switches, in order to change modes. As described above, the insertion speed of the needles can be controlled by the selected mode.

The gun has functions of directly inserting the microneedle mount on which the needles arc mounted into the human skin and pressing drugs supplied to the syringe connected to the microneedle mount. The gun has a shape of a handgun, a handgrip of the gun has an ON/OFF switch in a shape of a trigger, and a muzzle of the gun is provided with a bracket to insert the microneedle mount.

Therefore, after inserting and fixing the microneedle mount to the muzzle of the gun and pressing the switch, the microneedle mount is moved forward to enable the needles of the microneedle mount to pierce holes in the human skin, and when the syringe is pressed by continuous operations of the gun, the drugs in the syringe can be injected into the human skin through the microneedle mount and the needles.

Here, the gun can apply a general gun used for the microneedle therapy and the mesotherapy treatment.

In addition, in association with the mainboard control operations of the cart, a method of controlling the insertion speed and the insertion depth of the needles according to the selected mode, a method of controlling the operations of the gun, and the like are not limited and may employ any method well known in the art.

Hereinafter, the microneedle therapy system and method according to an embodiment of the present invention will be described in detail with reference to the attached drawings.

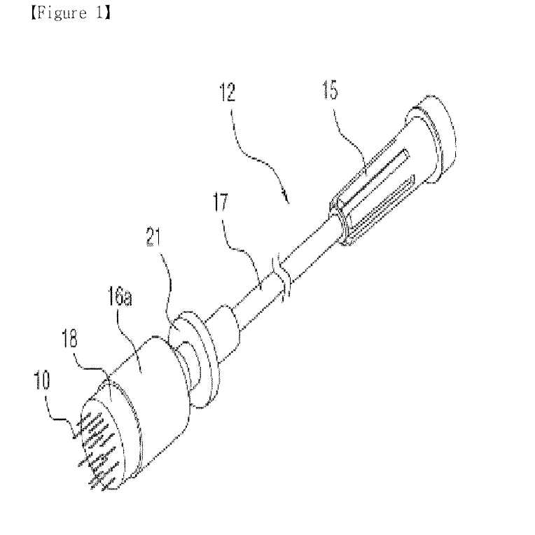

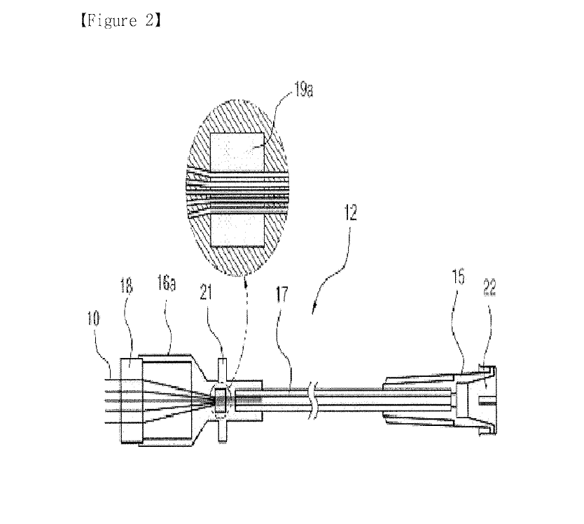

FIG. 1 is a perspective view illustrating a microneedle therapy system according to an embodiment of the present invention, and FIG. 2 is a cross-sectional view illustrating the microneedle therapy system according to the embodiment of the present invention.

Referring to FIGS. 1 and 2, a microneedle mount 12 having a number of needles 10 is exemplified.

The microneedle mount 12 is connected to a syringe 11 to have a structure for drug delivery and includes a part connected to the syringe 11, a part mounted to a gun, a tube part having a predetermined length, and a part to be inserted tinder the human skin. When the gun is operated, the microneedle mount 12 is supplied with drugs from the syringe to pierce a number of holes in the human skin through the needles and simultaneously inject the drugs.

For this, the microneedle mount 12 includes a syringe connector 15 in a shape of a cylinder to be connected with the syringe, a gun connector 16 which has a mounting portion 21 having a shape of a flange to be inserted and supported in a detachable structure and is mounted on the gun, a drug delivery tube 17 connected between the syringe connector 15 and the gun connector 16, and a number of needles 10 provided to a hub 18 engaged with a front portion of the gun connector 16.

The syringe connector 15 may be connected with the syringe so that the syringe connector 15 accommodates a part of a front portion of the syringe, and a rear portion of the syringe connector 15 includes a pad 22 into which a drug discharge portion of the syringe is inserted. When the syringe connector 15 is connected with the syringe, a needle of the syringe may be inserted into the syringe connector 15 through the pad 22.

The needles 10 are mounted to be inserted through the hub 18 and the gun connector 16 so as to be extended inside the drug delivery tube 17.

Accordingly, drugs supplied to the drug delivery tube 17 can be directly supplied to the needles 10 and injected into the human skin.

In addition, at the portion of the gun connector 16 through which the needles penetrate, a sealing portion 19a for preventing drug leakage is formed, and a sealing process can apply a conventional method.

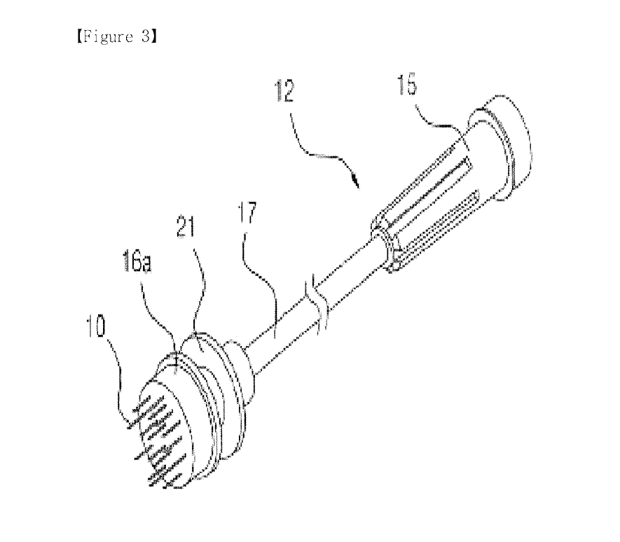

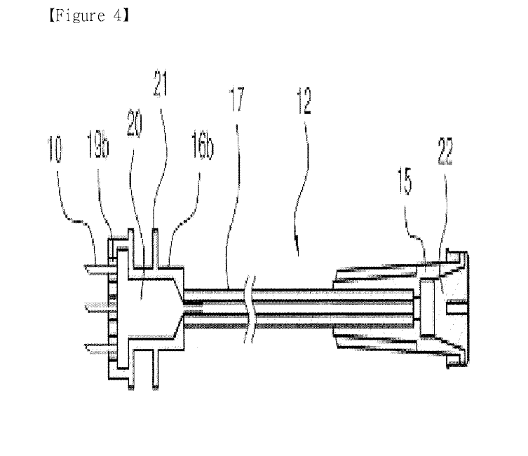

FIG. 3 is a perspective view illustrating a microneedle therapy system according to another embodiment of the present invention, and FIG. 4 is a cross-sectional view illustrating the microneedle therapy system according to the embodiment of the present invention.

Referring to FIGS. 3 and 4, another example of the microneedle mount 12 having a number of the needles 10 is illustrated. In this embodiment, the microneedle mount 12 has similar basic structures and functions to the microneedle mount described above and has some different shapes such as a mounted shape of the needles.

The microneedle mount 12 includes a syringe connector 15 having a pad 22 for connection to the syringe, a gun connector 16b to be mounted to the gun, a drug delivery tube 17, a number of needles 10, and the like.

Particularly, the gun connector 16b is provided with a hollow 20 so that the needles 10 mounted to be directly inserted into a front portion of the gun connector 16b can be connected with the hollow 20.

Accordingly, drugs supplied to the hollow 20 through the drug delivery tube 17 can be supplied to the needles 10 to be injected into the human skin.

Here, at the portion of the gun connector 16b through which the needles 10 penetrate, that is, at a front portion of the gun connector 16b through which the needles 10 are inserted, a sealing portion 19b is formed to prevent drug leakage.

The present invention provides a means for preventing drug loss in association with drug injection.

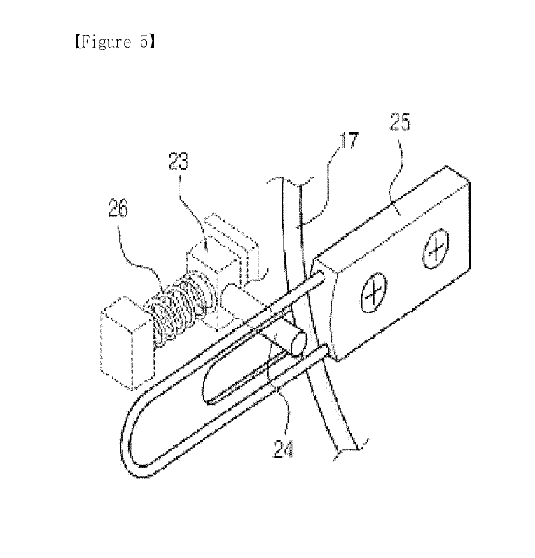

For this, as illustrated in FIG. 5, there is provided a drug stopper including a plate 23 which is provided inside a body of the gun and mounted on a rod of a cylinder (not shown) to insert the microneedle mount and the syringe to be moved along with the cylinder back and forth, a bar 24 which protrudes from the gun body and is disposed in front of the plate 23 to be moved by the contacting plate 23 back and forth to close or open the tube, a bracket 25 which faces the bar 24 with the interposed tube to support a side of the tube, and a spring 26 which is disposed in front of the bar 24 to provide restoring force to the bar 24.

Accordingly, in a state before the gun is operated, the drug delivery tube 17 is pressed between the bar 24 and the bracket 25 and closed, so that the drugs do not leak.

In a state where the gun is operated, that is, in a state where the microneedle mount is moved forward and inserted into the skin, the bar 24 is pushed forward by the plate 23 and separated from the tube 17, and the closed tube is opened to inject drugs.

Specifically, the tube is opened before the needles of the microneedle mount are inserted into the skin, so that drug injections are not interrupted.

After syringe procedures, the bar is returned to its original place to press and close the tube.

A treatment method using the microneedle therapy system having the aforementioned construction according to the present invention is described as follows.

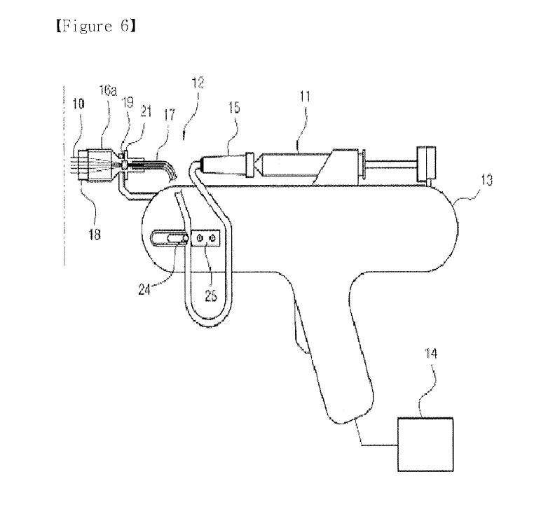

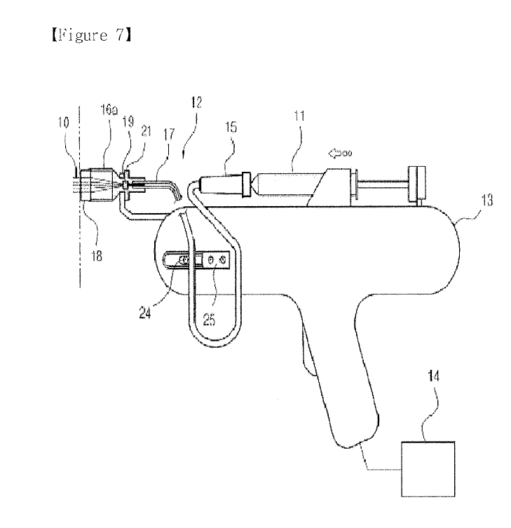

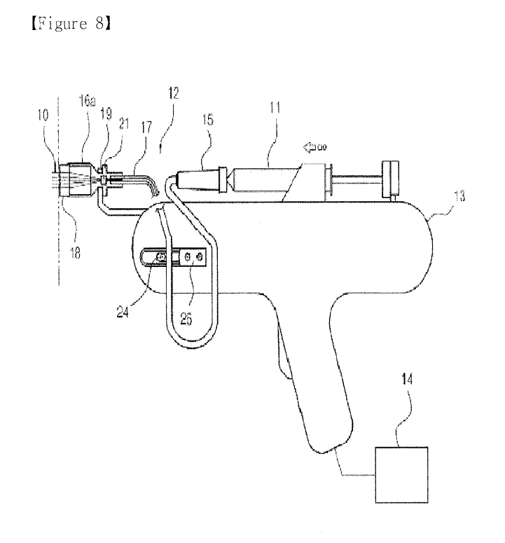

FIGS. 6 to 8 are schematic views illustrating operation states of the gun in the microneedle therapy system according to the embodiment of the present invention.

Referring to FIGS. 6 to 8, a microneedle therapy method basically employs operations of mounting the microneedle mount having a number of the needles to a gun that enables a linear motion, inserting the needles of the microneedle mount to be perpendicular to the skin by operating the gun, and injecting drugs supplied to the syringe through the needles.

In this method, the needles can be inserted in a predetermined depth at a predetermined speed.

First, in a preparation step, a power supply is connected, a cart and the gun are connected, a power switch is turned on, a mode switch is operated to set a mode according to characteristics of a patient, and the microneedle mount and the syringe which are connected to each other are mounted on the gun to be used.

Here, a stroke of the gun is set according to the selected mode, so that an insertion depth of the needles can be controlled.

When a gun switch is pushed after putting the end portion of the microneedle mount 12 mounted to a gunpoint of the gun, that is, the needles 10 to the skin to be treated, with a first step of the gun, the microneedle mount 12 and the syringe 11 are moved forward so as to enable the needles 10 to pierce the skin to create holes, and with a second step of the gun, a plunger of the syringe 11 is pressed to supply drugs in the syringe to the microneedle mount to be injected into the skin through the needles 10.

As the aforementioned steps are repeated, hundreds to ten thousands of holes can be created on the human skin and simultaneously the drugs can be injected into the skin.

As described above, the microneedle therapy method provided according to the present invention is a method of simultaneously forming channels on the epidermis and injecting drugs, so that a number of channels are immediately formed on the epidermis, and simultaneously the drugs can be injected. Therefore, as compared with conventional manual operations, treatment risks can be reduced.

In addition, since the needles are injected into the skin vertically, pain can be reduced by 90% as compared with conventional rollers. In addition, since the insertion depth of the needles can be controlled, deep scars or the like can be effectively treated.

In addition, by mounting a number of needles mounted to the microneedle mount, for example, by mounting 9 needles in a case where a diameter of the microneedle mount is 10 mm, narrow sites can be treated.

### INDUSTRIAL APPLICABILITY

The present invention has advantages in that a number of the needles are inserted into the human skin to create holes and simultaneously inject drugs, so that a procedure time can be reduced, pain during the treatment can be minimized, and drug absorption efficiency can be increased.

In addition, according to types of drugs, in addition to the microneedle therapy, the mesotherapy treatment requiring a number of injections can be performed for a short time.

In addition, a depth of a hole and the number of injections into the skin per minute can be controlled, so that the treatment effectiveness can be increased.

In addition, a price of a disposable needle is decreased as compared with a roller, and a simpler operating device is used as compared with a laser device. Therefore, the microneedle therapy and the mesotherapy treatment can be implemented at lower costs.

In addition, the treatment can be performed at deep sites in addition to narrow sites, so that the treatment can be performed according to sites.

1. A multi-injection microneedle therapy system comprising:
a microneedle mount which has a number of needles to deliver drugs and is connected to a syringe;
a gun which has functions of inserting the microneedle mount into the skin and injecting the drugs supplied to the syringe; and
a cart which has functions of providing power for operating the gun and simultaneously controlling the operations of the gun,
wherein a number of holes are created on the human skin and simultaneously the drugs are injected into the human skin by using the microneedle mount and the syringe according to the operations of the gun.

2. The multi-injection microneedle therapy system of claim 1, wherein the microneedle mount comprises:
a syringe connector connected with the syringe;
a gun connector mounted on the gun;
a drug delivery tube for connecting the syringe connector to the gun connector;
a plurality of the needles connected to a front portion of the gun connector; and
a hub for supporting the needles,
wherein the drugs supplied through the syringe connector are injected through the needles of the gun connector.

3. The multi-injection microneedle therapy system of claim 1 or 2, wherein the needles are mounted to be inserted through the gun connector so as to be extended inside the drug delivery tube to be directly supplied with the drugs from the drug delivery tube.

4. The multi-injection microneedle therapy system of claim 2, wherein a sealing portion for preventing drug leakage is formed at the portion of the gun connector through which the needles penetrate.

5. The multi-injection microneedle therapy system of claim 1, wherein the microneedle mount comprises:
a syringe connector connected with the syringe;
a gun connector which has a hollow through which the drugs are injected and is mounted on the gun;
a drug delivery tube for connecting the syringe connector to the gun connector; and
a plurality of the needles which penetrate a front portion of the gun connector to be engaged therewith and are connected with the hollow,
wherein the drugs supplied through the syringe connector are injected through the needles of the gun connector.

6. The multi-injection microneedle therapy system of claim 5, wherein a sealing portion for preventing drug leakage is formed at the portion of the gun connector through which the needles penetrate.

7. The multi-injection microneedle therapy system of claim 1, wherein the gun comprises a drug stopper comprising:
a plate which is provided inside of a body of the gun and mounted on a rod of a cylinder (not shown) to inject the microneedle mount and the syringe to be moved along with the cylinder back and forth;
a bar which protrudes from the body of the gun and is disposed in front of the plate to be moved by the contacting plate back and forth to close or open the tube;
a bracket which faces the bar with the interposed tube to support a side of the tube; and
a spring which is disposed in front of the bar to provide restoring force to the bar,
wherein the drug stopper closes the tube when the gun is not operated and releases the closed state of the tube when the gun is operated.
